(19) United States
(12) Patent Application Publication
(10) Pub. No.: US 2024/0261041 A1
(43) Pub. Date: Aug. 8, 2024

(54) APPARATUS FOR PREVENTING BUCKLING OF FLEXIBLE SURGICAL INSTRUMENT

(71) Applicants: ROEN Surgical, Inc., Daejeon (KR); KOREA ADVANCED INSTITUTE OF SCIENCE AND TECHNOLOGY, Daejeon (KR)

(72) Inventors: Dong-Soo KWON, Daejeon (KR); Hyun-Woo BAEK, Daejeon (KR); Joon-Yeong KIM, Daejeon (KR); Jae-Min YOU, Daejeon (KR); Duk-Yoo KONG, Daejeon (KR)

(73) Assignees: ROEN Surgical, Inc., Daejeon (KR); KOREA ADVANCED INSTITUTE OF SCIENCE AND TECHNOLOGY, Daejeon (KR)

(21) Appl. No.: 18/566,873

(22) PCT Filed: May 31, 2022
(86) PCT No.: PCT/KR2022/007723

§ 371 Date: Jan. 4, 2024

(30) Foreign Application Priority Data
Jun. 4, 2021 (KR) 10-2021-0072462

(51) Int. Cl. A61B 34/30

(52) U.S. Cl. CPC A61B 34/30; A61B 2034/301

(57) ABSTRACT

The present invention relates to an apparatus for preventing buckling of a flexible surgical instrument, and more particularly, to an apparatus for preventing buckling of a flexible surgical instrument for preventing the buckling when a catheter or an over-tube of a surgical instrument and the like is inserted into the human body. To this end, disclosed is a surgical-instrument feeder for preventing the buckling of flexible surgical instruments which comprises: a master driving unit for receiving a driving command from the user; and a feeder unit for feeding surgical instruments according to whether to interlock with the master driving unit or not interlock with the master driving unit, and feeding the surgical instruments supplied from the master driving unit so as to prevent the surgical instruments from buckling by means of the feeding control command.

### TECHNICAL FIELD

The present invention relates to an apparatus for preventing the buckling of flexible surgical instruments and, more specifically, to an apparatus for preventing buckling of a flexible surgical instrument which prevents the buckling phenomenon when a catheter or an overtube of a surgical instrument is inserted into a human body.

### BACKGROUND ART

Disclosed in Korean Patent Registration No. 10-2184889, which is a prior document, is a roller module for a medical robot, which is hygienic and can precisely transport a catheter, a driving device mounted on the medical robot, and a roller module mounted on the driving device.

At this time, the technology disclosed in the prior document uses five motors to drive forward/backward and to rotate the catheter, and two roller modules to move the catheter are used to always move the entire module up and down during sliding, thereby complicating the feature of the apparatus.

### DISCLOSURE

### Technical Problem

Accordingly, the present invention has been made keeping in mind the above problems occurring in the prior art, and an object of the present invention is to provide an apparatus for preventing a buckling phenomenon from occurring by disposing a roller module and a feeding mechanism for feeding a flexible surgical instrument to a human body as close as possible to the human body.

Another object of the present invention is to provide an invention in which a feature of a two-degree-of-freedom (translational motion and rotational motion) feeding mechanism is small and compact.

Further, another object of the present invention is to provide an invention in which a feeding mechanism is simple and easily detachable since a roller module must be disposable.

Further, the present invention has an object to provide an invention in which a drape is considered.

However, the objects of the present invention are not limited to the above-mentioned objects, and other objects not mentioned may be clearly understood by the skilled person in the art from the following description.

### Technical Solution

The purpose of the present invention can be achieved by providing a surgical instrument feeder apparatus for preventing the buckling of flexible surgical instruments, the apparatus comprising: a master driving unit for receiving a driving command from a user; and a feeder unit for feeding surgical instruments according to whether to interlock with the master driving unit or not interlock with the master driving unit, and feeding the surgical instruments supplied from the master driving unit so as to prevent the surgical instruments from buckling by means of the feeding control command.

In addition, the apparatus further includes a connection support unit mechanically connecting and supporting the master driving unit and the feeder unit.

The feeder unit includes a plurality of rollers disposed parallel to each other on a plurality of roller driving shafts while being disposed at an end of the connection support unit, which is being separated from the master driving unit by a predetermined distance, and the plurality of rollers is disposed near the affected area to translate and rotate the surgical instrument, thereby preventing buckling of the surgical instrument.

Also, the feeder unit includes: a plurality of roller-driving shafts disposed in parallel to each other; a plurality of roller-rotation driving units in which the plurality of driving shafts interlock with each other by means of a gear to rotate in different directions so that the surgical instrument translates; and a roller-horizontal-movement driving unit in which a driving force is generated to horizontally move the plurality of roller-driving shafts in different directions and transmitted to the driving shaft to rotate the surgical instrument.

Further, the roller driving shaft and the driving shaft are coupled together such that the driving shaft and the driving shaft are attachable/detachable in a horizontal direction, to thereby prevent mechanical restraint.

In addition, the plurality of roller rotation driving units rotate the plurality of roller driving shafts in different directions by interlocking operation of one motor and a gear.

Also, a driving shaft of the roller rotation driving unit is disposed on a roller driving shaft to transmit rotational driving force such that the roller driving shaft rotates in different directions, and a driving shaft of the roller horizontal movement driving unit is disposed perpendicular to the driving shaft of the roller rotation driving unit to transmit horizontal movement driving force to the driving shaft such that the roller driving shaft horizontally moves in different directions.

Meanwhile, an object of the present invention is to provide a surgical instrument including a feeder driving unit generating driving force to allow a surgical instrument to be moved in translation and to rotate, a driving shaft coupling module unit which is first coupled with the driving shaft of the feeder driving unit in a horizontal direction to be mechanically detachable, a roller module unit which is second coupled with the coupling shaft of the driving shaft coupling module unit in a horizontal direction to be mechanically detachable, and an elasticity providing unit providing elasticity to maintain mechanical coupling between the first and second couplings which are mutually restrained to be mechanically detachable.

In addition, a translation gear shaft of a feeder driving unit for translating the surgical instrument and a rotation gear shaft of the feeder driving unit for rotating the surgical instrument are disposed perpendicularly to each other, and the surgical instrument is disposed in a space between a plurality of rollers of the roller module unit while being disposed in parallel to the rotation gear shaft.

In addition, the feeder driving unit includes: a plurality of roller rotation driving units which generates rotation driving force to rotate a plurality of gear driving shafts arranged in parallel to each other in different directions by gear engagement to rotate the plurality of roller rotation shafts mechanically coupled to the plurality of gear driving shafts in different directions, thereby translating the surgical instrument; and a roller horizontal movement driving unit which generates horizontal movement driving force to horizontally move the plurality of gear driving shafts in different directions by being vertically arranged to the plurality of gear driving shafts to horizontally move the plurality of gear driving shafts in different directions, thereby rotating the surgical instrument by horizontally moving the plurality of roller rotation shafts mechanically coupled to the plurality of gear driving shafts in different directions.

Further, the plurality of roller rotation driving units includes a first roller rotation driving unit which is firstly coupled to the driving shaft coupling module unit in the horizontal direction, and a second roller rotation driving unit which is arranged in parallel to the first roller rotation driving unit and is secondly coupled to the driving shaft coupling module unit in the horizontal direction.

In addition, the first roller rotation driving unit includes: a first gear driving shaft rotating according to a rotation driving force; a first gear unit rotating according to the rotation of the first gear driving shaft; a bearing unit rotating the first gear unit and relatively rotationally restrained to horizontally move in the first direction together with the first gear driving shaft according to the transmission of a first direction horizontal movement driving force; and a roller rotation motor unit generating a rotation driving force to rotate the first gear driving shaft.

In addition, the second roller rotation driving unit includes: a second gear unit gear-meshed with the first gear unit to rotate in a direction different from the rotation direction of the first gear unit; a second gear driving shaft rotating in the same direction as the rotation direction of the second gear unit; a bearing unit rotating the second gear unit and restrained from rotating, and horizontally moving together with the second gear driving shaft in the second direction according to the transmission of the second direction horizontal movement driving force; and a motor coupler unit having a driving shaft insertion movement groove into which the second gear driving shaft is inserted to horizontally move in the second direction.

Further, the roller horizontal movement driving unit includes a pinion unit configured to rotate with respect to the pinion rotation shaft by a horizontal movement driving force, a first rack unit engaged with a gear of the pinion unit to generate a first direction horizontal movement driving force, a second rack unit engaged with a gear of the pinion unit to generate a second direction horizontal movement driving force, and a roller horizontal movement motor unit configured to generate a horizontal movement driving force to rotate the pinion rotation shaft.

Further, the present invention further includes: a first driving shaft horizontal movement coupling unit which is coupled to the first rack unit and the bearing unit of the first roller rotation driving unit, respectively, to transfer a first direction horizontal movement driving force to the bearing unit of the first roller rotation driving unit and restrains the rotation of the bearing unit of the first roller rotation driving unit; a second driving shaft horizontal movement coupling unit which is coupled to the second rack unit and the bearing unit of the second roller rotation driving unit, respectively, to transfer a second direction horizontal movement driving force to the bearing unit of the second roller rotation driving unit and restrains the rotation of the bearing unit of the second roller rotation driving unit; a first rail unit which allows the first driving shaft horizontal movement coupling unit to move in a horizontal direction; and a second rail unit which allows the second driving shaft horizontal movement coupling unit to move in a horizontal direction.

The driving shaft coupling module unit includes a first coupling disk unit which is firstly coupled to the first gear driving shaft of the feeder driving unit in a horizontal direction and is secondly coupled to the first roller rotation shaft of the roller module unit in the horizontal direction, and a second coupling disk unit which is arranged in parallel to the first driving shaft, is firstly coupled to the second gear driving shaft of the feeder driving unit in the horizontal direction, and is secondly coupled to the second roller rotation shaft of the roller module unit in the horizontal direction.

Further, the roller module unit comprises: a first surgical instrument roller unit which is secondarily coupled to one side of the first coupling disk unit, and which is elastically supported by a first elastic unit of the elasticity providing unit and the other side thereof; and a first surgical instrument roller unit which is secondarily coupled to one side of the second coupling disk unit, and which is elastically supported by a second elastic unit of the elasticity providing unit and the other side thereof.

In addition, the plurality of rotation shafts of the roller module unit and the plurality of gear driving shafts of the feeder driving unit are respectively and mechanically restrained so as to be attachable and detachable on a shaft by first and second coupling disk units, and are first and second coupled, thereby translating or rotating the surgical instrument.

The apparatus may further include a surgical instrument guide unit to guide the surgical instrument supplied from the master driving unit to a space between the plurality of rollers of the roller module unit.

Also, the surgical instrument guide unit comprises: a guide body formed with a surgical instrument penetration hole; a body coupling unit for coupling and fixing the guide body to the driving shaft coupling module unit and the elasticity providing unit, respectively; and a tubular guide unit for guiding the surgical instrument.

In addition, the latch assembly further includes an attachable/detachable fixing latch unit which releases the restraining of the driving shaft coupling module unit mechanically restrained and fixed by moving the latch by an external force and mechanically restrains and fixes the driving shaft coupling module unit by returning to the original position of the latch as the external force is removed.

The driving shaft coupling module further includes a protection cover unit protecting the first and second coupling disk units and having a latch insertion groove into which the latch is inserted and restrained.

The feeder driving unit comprises: a protection cover coupling plate in which an L-shaped protection cover holding unit is formed so as to hold a plurality of edges of the protection cover unit and in which a latch penetration groove is formed so as to assist in attaching, detaching, restraining and fixing the latch unit; and a latch coupling plate unit to which the attaching, detaching, and fixing latch unit is coupled and fixed, together with the protection cover coupling plate, as a housing of the feeder driving unit.

In addition, the detachable fixing latch unit includes a push button unit to which an external force is applied, a latch medium unit which rotates at a predetermined angle around a rotation shaft according to an elastic pressing of the push button unit, and a latch unit which elastically moves in a horizontal direction in conjunction with the rotation of the latch medium unit.

In addition, the latch unit horizontally and elastically moves in a direction opposite to a direction in which an external force is applied, thereby releasing restraint of the driving shaft coupling module unit.

Also, the latch medium unit is disposed to be inclined at a predetermined angle from the initial state in which the external force is removed.

Further, a restraining disk is circumferentially formed at one side of the first and second coupling disk units, and a restraining disk insertion groove is circumferentially formed at the other side of the first and second coupling disk units.

### Advantageous Effects

According to the present invention, the roller module and the feeding mechanism for feeding the flexible surgical instrument onto the human body are arranged as close to the human body as possible to prevent buckling.

Also, according to the present invention, there is the advantage that it is possible to provide a simple feeder mechanism wherein, when a surgical instrument is moved forwards/backwards, only upper and lower rollers that rotate in mutually different directions are rotated, and, when a surgical instrument is rotated, upper and lower driving shafts that have been mechanically restrained and engaged with a plurality of rollers are moved in mutually different directions in the horizontal direction, and hence there is no need to rotate or move the entire roller module.

### DESCRIPTION OF DRAWINGS

The following drawings attached to the present specification exemplify a preferred embodiment of the present invention and serve to further understand the technical spirit of the present invention together with the detailed description of the present invention, and thus the present invention should not be construed as being limited to the matters described in such drawings.

FIG. 1 is a view illustrating a master driving unit, a connection support unit, and a surgical instrument feeder unit according to an embodiment of the present invention

FIGS. 2 to 3 are views illustrating a surgical instrument guide unit according to an embodiment of the present invention

FIGS. 4 to 7 are views illustrating an elasticity supply unit 200 according to an embodiment of the present invention

FIGS. 8 to 10 are views illustrating a roller module unit according to an embodiment of the present invention

11 to 16 are views illustrating a driving shaft coupling module unit according to an embodiment of the present invention

FIG. 17 is a view illustrating a protective cover coupling plate according to an embodiment of the present invention

FIGS. 18 to 20 are views illustrating an upper roller rotation driving unit and a lower roller rotation driving unit according to an embodiment of the present invention

FIG. 21 is a view illustrating a horizontal movement of an upper gear driving shaft and a lower gear driving shaft according to an embodiment of the present invention in different directions

FIG. 22 to FIG. 26 are views illustrating the roller horizontal movement driving unit in accordance with an embodiment of the present invention

FIG. 27 is a view illustrating that upper and lower gear driving shafts and pinion rotation shafts (or pinion driving shafts) are disposed in perpendicular directions to each other in accordance with an embodiment of the present invention

FIG. 28 is a view illustrating a latch coupling plate unit in accordance with an embodiment of the present invention, and

FIG. 29 to FIG. 34 are views illustrating a detachable fixing latch unit in accordance with an embodiment of the present invention.

### MODE FOR INVENTION

Hereinafter, a preferred embodiment of the present invention will be described with reference to the drawings. In addition, the exemplary embodiment described below does not unreasonably limit the contents of the present invention described in the claims, and it cannot be said that the entire feature described in the present embodiment is essential as a means for solving the present invention. Further, descriptions of the prior art and the skilled person in the art may be omitted, and descriptions of the omitted elements (methods) and functions may be fully referred to without departing from the technical spirit of the present invention.

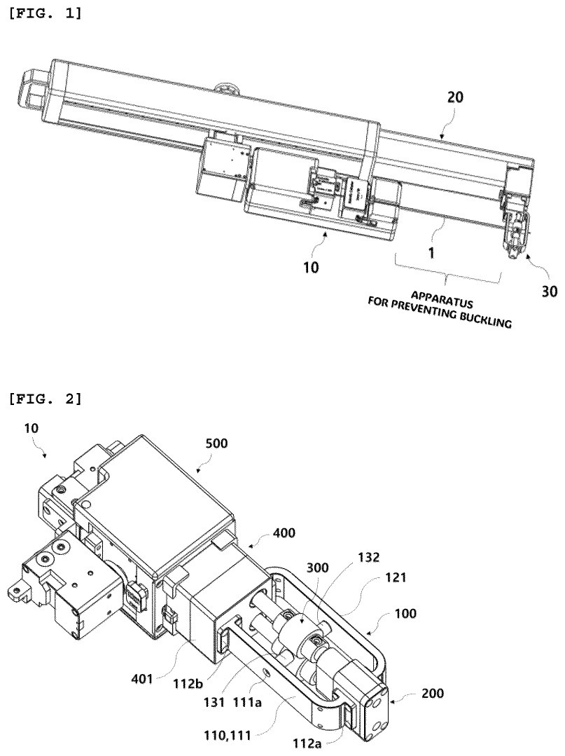

As shown in FIG. 1, the apparatus for preventing buckling of a flexible surgical instrument (hereinafter, referred to as a surgical instrument feeder apparatus) according to an embodiment of the present invention includes a master driving unit 10, a connection support unit 20, and a surgical instrument feeder unit 30 to insert the surgical instrument 1 into the human body. The surgical instrument 1 is supplied from the master driving unit 10 to the surgical instrument feeder unit 30 according to a manipulation command of the master driving unit 10, and the surgical instrument feeder unit 30 includes a separate roller module 300 to feed the supplied surgical instrument 1 as close to the human body as possible.

Meanwhile, the buckling phenomenon refers to a phenomenon in which, when a surgical robot inserts a surgical instrument into a human body, the surgical instrument is not further inserted into the human body and is not moved by a forward movement control command of a robot driving unit. The surgical instrument feeder apparatus according to the present invention, as shown in FIG. 1, has an advantage in that the surgical instrument feeder unit 30 feeds the surgical instrument 1 as close to the human body as possible and a separate surgical instrument feeding mechanism is implemented to prevent the occurrence of buckling.

As shown in FIG. 1, the master driving unit 10 generates an overall manipulation control command for driving the surgical instrument feeder apparatus as the master apparatus of the present invention, and supplies the surgical instrument 1 to the surgical instrument feeder unit 30 when a surgical instrument insertion control command is received from the user.

The surgical instrument 1 of the present invention is a flexible surgical instrument to be inserted into a human body, and includes all surgical instruments to be inserted into a human body, such as a catheter or an overtube, and is commonly referred to as a surgical instrument in the present invention.

In order to dispose the surgical instrument feeder unit 30 as close to the human body as possible, the master driving unit 10 and the surgical instrument feeder unit 30 are respectively supported and coupled to the connection support unit 20. The master driving unit 10 is fixedly disposed at one side of the connection support unit 20, and the surgical instrument feeder unit 30 is fixedly disposed at the other side of the connection support unit 20. In this case, as shown in FIG. 1, the surgical instrument 1 is disposed in a space between the master driving unit 10 and the surgical instrument feeder unit 30, and the surgical instrument feeder unit 30, in which a feeding mechanism is implemented to prevent buckling in the space between the master driving unit 10 and the surgical instrument feeder unit 30, is disposed and fixed at one end of the connection support unit 20. The surgical instrument 1 is supplied from the master driving unit 10 to the surgical instrument feeder unit 30, and the surgical instrument feeder unit 30 feeds the surgical instrument 1 into the human body while preventing a buckling phenomenon by using a separate feeding mechanism.

Meanwhile, when the surgical instrument 1 is supplied to the surgical instrument feeder unit 30 according to the control operation of the master driving unit 10, the roller module unit 300 of the surgical instrument feeder unit 30 interlocks with the master driving unit 10 to insert the supplied surgical instrument 1 into the human body. This operation may be implemented in an interworking operation mode. On the other hand, when the buckling phenomenon occurs due to the interlocking operation, the roller module unit 300 of the surgical instrument feeder unit 30 may be driven alone according to the non-interlocking operation mode, thereby solving the buckling phenomenon. Here, the interoperation operation mode is a mode in which the master surgical instrument supply unit of the master driving unit 10 and the roller module unit 300 of the surgical instrument feeder unit 30 interoperate with each other to insert the surgical instrument 1 into the human body, and the non-interoperation operation mode is a single driving mode of the roller module unit 300 of the surgical instrument feeder unit 30.

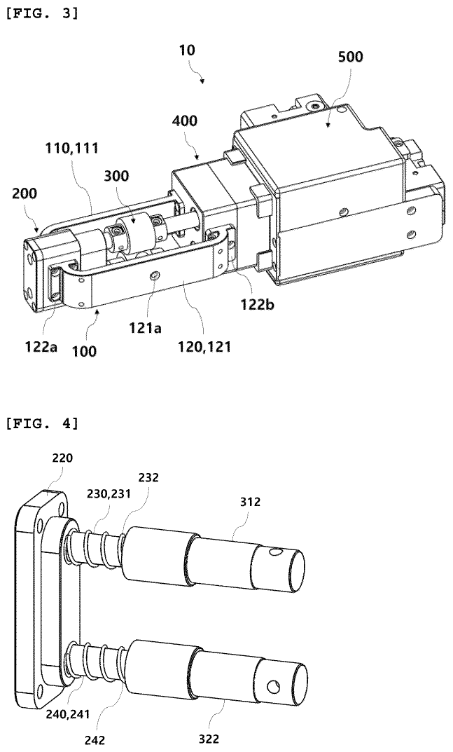

As shown in FIGS. 2 and 3, the surgical instrument guide unit 100 according to an embodiment of the present invention guides the surgical instrument 1 supplied from the master surgical instrument supply unit of the master driving unit. To this end, first and second surgical instrument guide units 110 and 120 and first and second tubular guide units 131 and 132 are included.

As shown in FIG. 2, the first surgical instrument guide unit 110 includes a guide body 111, first and second body coupling units 112a and 112b, and a first tubular guide unit 131. A surgical instrument penetration hole 111a is formed in a central region of the guide body 111, and the surgical instrument 1 supplied from the master surgical instrument supply unit of the master driving unit 10 passes therethrough. First and second body coupling units 112a and 112b are formed at both ends of the guide body 111, respectively. The first body coupling unit 112a is coupled to one side of the body of the elasticity providing unit 200. The second body coupling unit 112b is coupled to one side of the first protection cover unit 401 of the driving shaft coupling module 400. The first tubular guide unit 131 communicates with the surgical instrument penetration hole 111a and is disposed and coupled in the traveling direction of the surgical instrument 1. Accordingly, the surgical instrument 1 supplied from the master surgical instrument supply unit is guided along the first tubular guide unit 131 and is fed to the space between the roller units 311 and 321 that face each other and are disposed in pairs.

The second surgical instrument guide unit 120 includes a guide body 121, first and second body coupling units 122a and 122b, and a second tubular guide unit 132. The guide body 121 has a surgical instrument penetration hole 121a formed at a central region thereof, and the surgical instrument 1 supplied from the first tubular guide unit 131 is guided along the second tubular guide unit 132 and finally passes. The passed surgical instrument 1 is inserted into the human body. First and second body coupling units 122a and 122b are formed at both ends of the guide body 121, respectively. The first body coupling unit 122a is coupled to the other side of the body of the elasticity providing unit 200. The second body coupling unit 122b is coupled to the other side of the first protection cover unit 401 of the driving shaft coupling module 400. The second tubular guide unit 132 communicates with the surgical instrument penetration hole 121a, and is disposed and coupled in the traveling direction of the surgical instrument so as to face the first tubular guide unit 131.

As shown in FIGS. 2 and 3, the guide body 111 of the first surgical instrument guide unit 110 and the guide body 121 of the second surgical instrument guide unit 120 are disposed to face each other, and the first and second tubular guide units 131 and 132 and the roller module unit 300 are disposed in a space between the guide bodies 111 and 121. As shown in FIG. 2, the first and second tubular guide units 131 and 132 are disposed to face each other, and feed the surgical tool 1 into a space between the roller units 311 and 321 that are disposed in pairs while facing each other up and down.

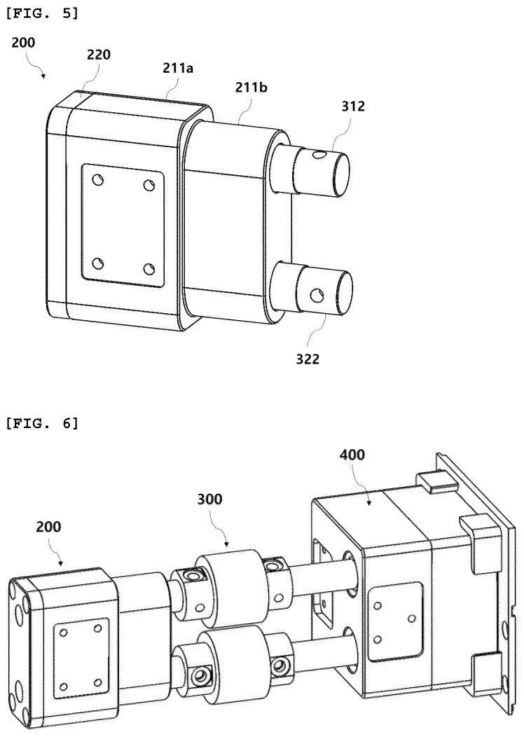

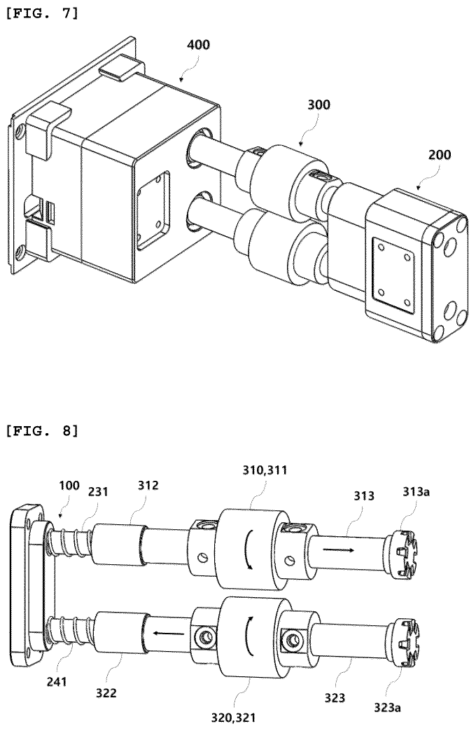

As shown in FIGS. 4 to 7, the elasticity providing unit 200 according to an embodiment of the present invention includes a first elastic unit 230 and a second elastic unit 240 which are disposed in parallel to each other. The first elastic unit 230 includes a spring insertion shaft 231 and a spring 232. The pair of second elastic units 240 also include a spring insertion shaft 241 and a spring 242. As illustrated in FIG. 5, the first protection cover unit 211a protects the springs 232 and 242, and the second protection cover unit 211b protects the first roller rotation shafts 312 and 322. The first body coupling units 112a and 122a are coupled to both sides of the first protective cover unit 211a, respectively.

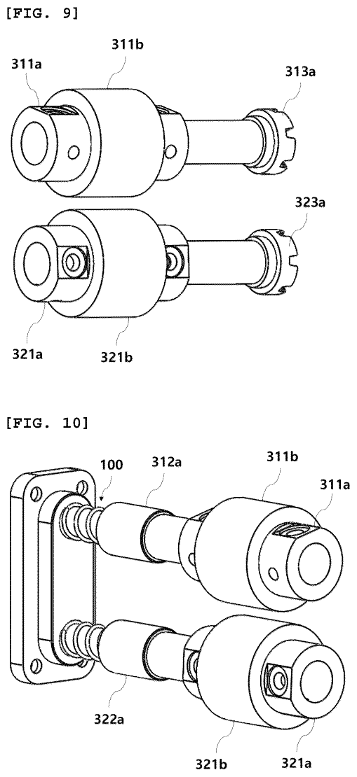

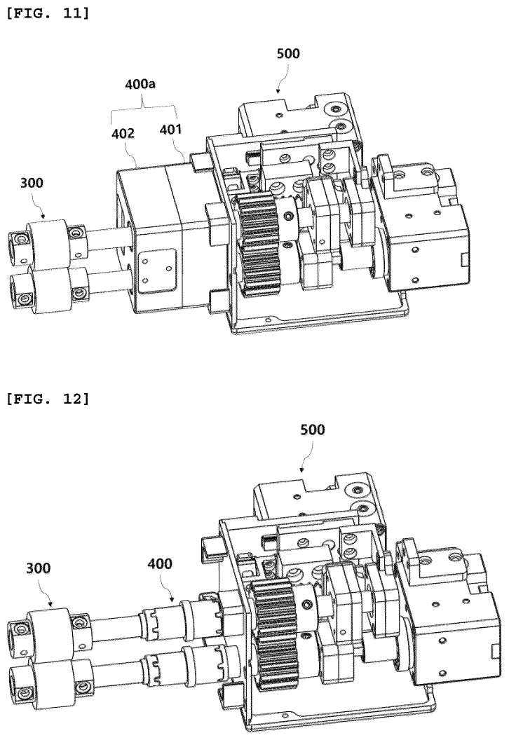

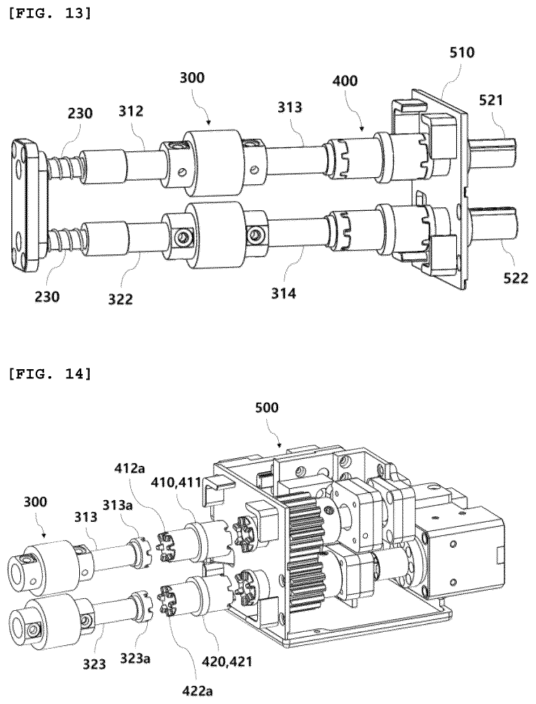

The spring insertion shafts 231 and 241 are supported and coupled to the spring support unit 220 at a predetermined distance from each other in the vertical direction, and the springs 232 and 242 are inserted into the spring insertion shafts 231 and 241, respectively, to provide elasticity to the first roller rotation shafts 312 and 322. As shown in FIGS. 6 and 7, the first roller rotation shafts 312 and 322 are elastically supported by the first and second elastic units 230 and 240, respectively, so that the driving shaft coupling module unit 400 mechanically restrained and coupled to the driving shaft coupling module unit can be detachably attached to the driving shaft coupling module unit while maintaining coupling.

The roll module unit 300 according to the exemplary embodiment of the present invention includes a first surgical instrument roller unit 310 and a second surgical instrument roller unit 320 that are disposed in parallel to each other in a pair.

The first surgical instrument roller unit 310 includes a roller unit 311 and first and second roller rotation shafts 312 and 313, and the second surgical instrument roller unit 320 includes a roller unit 321 and first and second roller rotation shafts 322 and 323.

The roller units 311 and 321 include roller support shafts 311a and 321a and rollers 311b and 321b, respectively. Each of the rollers 311b and 321b is coupled to each of the roller support shafts 311a and 321a. The surgical tool 1 guided by the surgical tool guide unit 100 is supplied to a space between the roller 311b and the roller 321b, and the surgical tools engaged by the rollers 311b and 321b may move straight or backward by rollers rotating in opposite directions. Here, the straight line is the direction in which the surgical instrument is inserted into the patient's body. The rotation directions of the rollers 311b and 321b illustrated in FIG. 8 are, for example, illustrated and rotate in opposite directions. In addition, the first and second roller rotation shafts 312 and 313 and the first and second roller rotation shafts 322 and 323 may be horizontally moved in different directions to rotate the surgical tool 1. The horizontal movement direction of the roller rotation shaft shown in FIG. 8 is shown as an example, and the surgical instrument may be rotated clockwise or counterclockwise by always moving in opposite directions.

One side of each of the first roller rotation shafts 312 and 322 is inserted into and coupled to each of the roller support shafts 311a and 321a, and the other side of each of the first roller rotation shafts 312 and 322 is inserted into and coupled to each of the spring insertion shafts 231 and 241. One side of each of the second roller rotation shafts 313 and 323 is inserted into and coupled to each of the roller support shafts 311a and 321a, and coupling disk coupling units 313a and 323a are formed on the other side of each of the second roller rotation shafts 313 and 323.

As shown in FIGS. 11 and 16, the driving shaft coupling module unit 400 according to the embodiment of the present invention includes first and second protective cover units 401 and 402 for protecting the coupling disk, and first and second coupling disk units 410 and 420.

The first and second protective cover units 401 and 402 are provided with grooves, through which the coupling disk body units 411 and 421 and the second roller rotation shafts 313 and 323 mechanically connected to and restrained by the coupling disk body units 411 and 421, respectively, may pass, in parallel to each other, and are formed to penetrate along the length of the body

The first protective cover unit 401 is provided with a latch coupling groove to which the latch body unit 631 is coupled and restrained. In addition, second body coupling units 112b and 122b are coupled to the second protective cover unit 402, respectively. A drape may be inserted between the first protective cover unit 401 and the second protective cover unit 402 for a clinical trial, etc.

As shown in FIGS. 13 to 16, the first coupling disk unit 410 includes a coupling disk body unit 411, and the second coupling disk unit 420 includes a coupling disk body unit 421. First coupling coupling members 412a and 422a and second coupling members 412b and 422b are formed at both ends of the coupling disk bodies 411 and 421, respectively, and mechanically restrained to each other.

The first coupling coupling units 412a and 422a are mechanically coupled to the coupling disk coupling units 313a and 323a, respectively, and are detachably restrained. In addition, the second coupling coupling coupling units 412b and 422b are mechanically coupled to the upper and lower coupling disk coupling units 521a and 531a, respectively, and are detachably restrained. The first coupling coupling units 412a and 422a and the coupling disk coupling units 521a and 531a respectively include restraint disks formed in circumferential directions, and the second coupling units 412b and 422b and the coupling disk coupling units 313a and 323a respectively include restraint disk insertion grooves formed in circumferential directions. The restraint disks are mechanically coupled by being inserted into and restrained in the restraint disk insertion grooves, respectively.

The upper driving shaft unit and the lower driving shaft unit, which mechanically connect and restrain the elasticity providing unit 200, the roller module unit 300, and the driving shaft coupling module unit 400, are arranged in pairs in parallel while being spaced apart from each other by a predetermined distance.

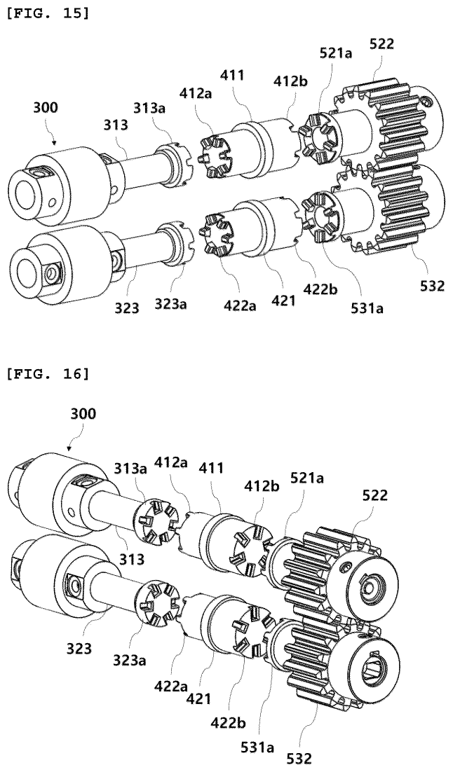

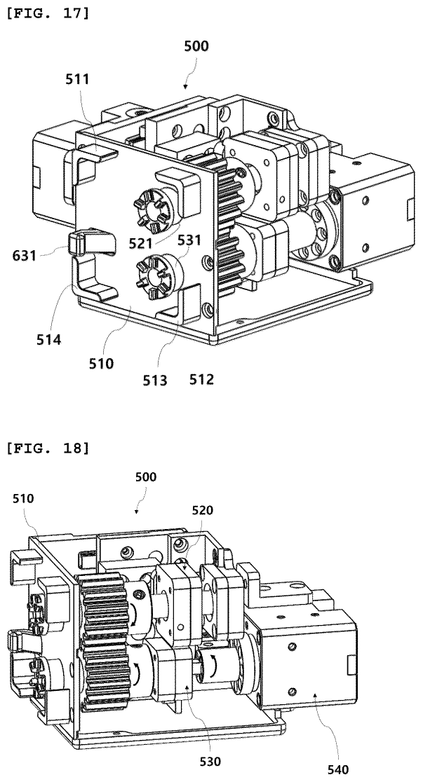

As shown in FIG. 17, the protection cover coupling plate 510 is a housing of the feeder driving unit 500, and has four protection cover holders 511, 512, 513, and 514 capable of supporting the protection cover body unit 400a on four surfaces in the circumferential direction. In addition, the protective cover coupling plate 510 is provided with a latch penetration groove through which the latch body units 631 may penetrate and a gear driving shaft penetration groove through which the first and second gear driving shafts 521 and 531 may penetrate.

As shown in FIGS. 17 to 26, the feeder driving unit 500 according to the embodiment of the present invention rotates the rollers 311b and 312b in different directions to move the surgical tool 1 forward or backward, and horizontally moves the upper driving shaft unit and the lower driving shaft unit in different directions to provide driving force for rotating tool 1 clockwise or the surgical counterclockwise.

As shown in FIGS. 18 and 19, the upper roller rotation driving unit 520 includes an upper gear driving shaft 521, an upper gear unit 522, and upper bearing units 523 and 524, and the lower roller rotation driving unit 530 includes a lower gear driving shaft 531, a lower gear unit 532, a lower bearing unit 533, and a motor coupler unit 534.

The upper and lower gear driving shafts 521 and 531 and the upper and lower gear units 522 and 532 are disposed in pairs in parallel with each other, and the upper gear unit 522 and the lower gear unit 532 are engaged with each other. Therefore, the upper gear unit 522 is gear-interlocked by the rotation of the lower gear unit 532 in the first direction to rotate in the second direction (the direction opposite to the first direction). FIG. 18 shows the rotation direction of each gear as an example, but the gears always rotate in opposite directions.

The upper gear unit 522 and the lower gear unit 532 are inserted into and coupled to the upper gear driving shaft 521 and the lower gear driving shaft 531, respectively. In addition, an upper coupling disk coupling unit 521a and a lower coupling disk coupling unit 531a are formed at one end of the upper gear driving shaft 521 and the lower gear driving shaft 531, respectively.

The lower bearing unit 533 and the motor coupler unit 534 are inserted into and coupled to the lower gear driving shaft 531. In addition, the first and second upper bearing units 523 and 524 are insertedly coupled to the upper gear driving shaft 521. Therefore, the lower gear unit 532, the lower bearing unit 533, and the motor coupler unit 534 are sequentially inserted and disposed based on the lower gear driving shaft 531. In addition, the upper gear unit 522, the first upper bearing unit 523, and the second upper bearing unit 524 are sequentially inserted and disposed with respect to the upper gear driving shaft 521.

The first upper bearing unit 523 and the second upper bearing unit 524 are restrained so as not to rotate by themselves, and allow the upper gear driving shaft 521 to rotate. The lower bearing unit 533 is restrained not to rotate by itself, and allows the lower gear driving shaft 531 to rotate.

One side of the motor coupler unit 534 is restrained and coupled to the roller rotation motor unit 540, and the other side is restrained and coupled to the lower gear driving shaft 531. Accordingly, when the roller rotation motor unit 540 provides a rotation driving force, the motor coupler unit 534 and the lower gear driving shaft 531 are rotated in conjunction with the rotation driving force. A driving shaft insertion movement groove 534a into which the lower gear driving shaft 531 is inserted and moved is formed at an inner center of the motor coupler unit 534. Therefore, a space in which the lower gear driving shaft 531 may horizontally move according to driving of the roller horizontal movement driving unit 550 to be described later is provided.

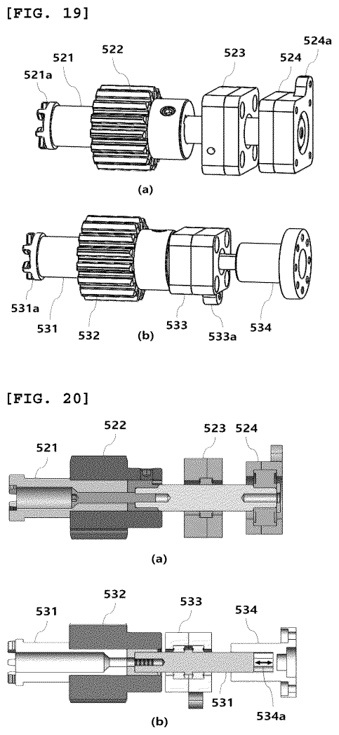

As shown in FIG. 21, the roller horizontal movement driving unit 550 according to an embodiment of the present invention generates horizontal movement driving force for horizontally moving the upper and lower gear driving shafts 521 and 531 in opposite directions.

As shown in FIGS. 22 to 26, the roller horizontal movement driving unit 550 includes a pinion unit 551, first and second rack units 552a and 552b, first and second driving shaft horizontal movement coupling units 553a and 553b, and first and second rail units 554a and 554b.

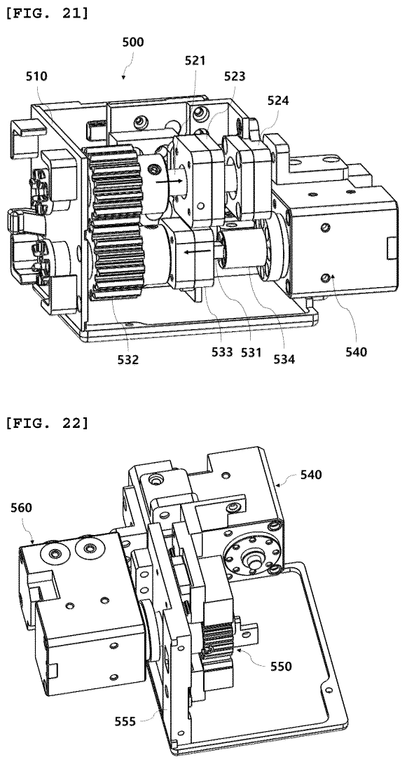

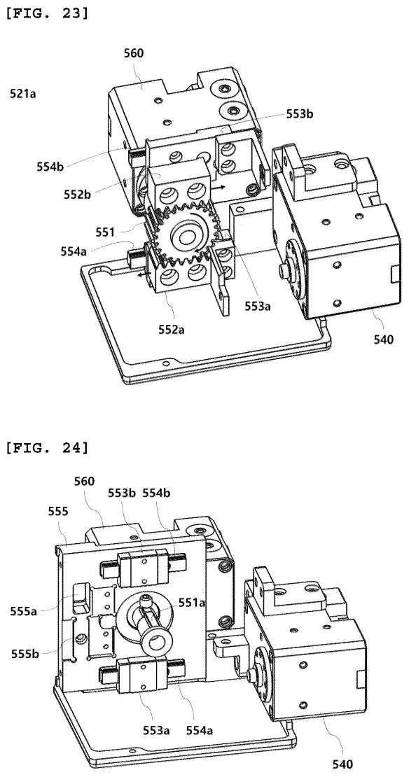

As shown in FIGS. 23 and 24, the pinion unit 551 is gear-engaged with each rack unit at the center of the first and second rack units 552a and 552b. In addition, the pinion unit 551 is inserted into and restrained by the pinion rotation shaft 551a and rotates according to the rotation of the pinion rotation shaft 551a. The pinion rotation shaft 551a rotates according to a horizontal movement driving force provided by the roller horizontal movement motor unit 560. The latch coupling plate unit 555 is a housing of the feeder driving unit 500 and has a through-groove through which the pinion rotation shaft 551a passes.

The first rack unit 552a is gear-engaged with the pinion unit 551 at the lower unit of the pinion unit 551, and the second rack unit 552b is gear-engaged with the pinion unit 551 at the upper unit of the pinion unit 551. Therefore, as illustrated in FIG. 23, when the pinion 551 rotates clockwise, the first rack 552a horizontally moves to the left and the second rack 552b horizontally moves to the right.

As shown in FIGS. 23 and 24, the first and second driving shaft horizontal movement coupling units 553a and 553b include a first coupling unit coupled to the first and second rack units 552a and 552b and a second coupling unit coupled to the first and second rail units 554a and 554b by rails, and the first and second coupling units are coupled to each other. In addition, the first and second rail units 554a and 554b are coupled to the latch coupling plate unit 555.

Meanwhile, when the pinion 551 is rotated in any one direction according to the horizontal movement driving force, the first rack 552a is horizontally moved in the first direction, and the second rack 552b is horizontally moved in the second direction different from the first direction. The second coupling unit of the first driving shaft horizontal movement coupling unit 553a coupled to the first rack unit 552a horizontally moves in the first direction along the first rail unit 554a, and the second coupling unit of the second driving shaft horizontal movement coupling unit 553b coupled to the second rack unit 552b horizontally moves in the second direction along the second rail unit 554b.

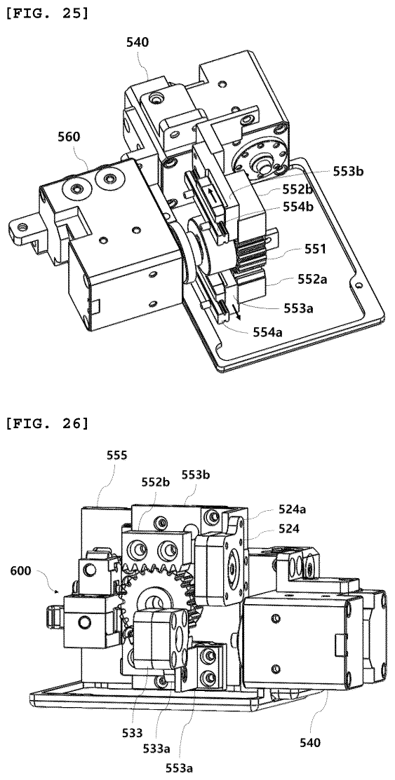

In addition, the first coupling unit of the first driving shaft horizontal movement coupling unit 553a is coupled to the first rack unit 552a and is coupled to the driving shaft horizontal movement coupling unit 533a of the lower bearing unit 533 as illustrated in FIG. 26. The first coupling unit of the second driving shaft horizontal movement coupling unit 553b is coupled to the driving shaft horizontal movement coupling unit 524a of the second upper bearing unit 524 while being coupled to the second rack unit 552b. The rotation of the lower bearing unit 533 and the second upper bearing unit 524 is restrained, and the lower bearing unit and the second upper bearing unit move horizontally along the horizontal movement directions of the first rack unit 552a and the second rack unit 552b, respectively.

The roller horizontal movement driving unit 550 may provide a driving force exceeding the elastic force of the elasticity providing unit 200 described above, and thus may horizontally move the upper driving shaft unit and the lower driving shaft unit in different directions to rotate the surgical instrument 1 clockwise or counterclockwise.

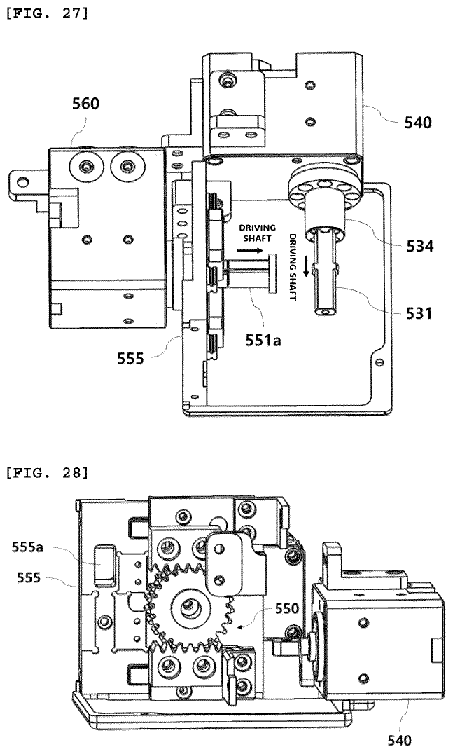

As illustrated in FIG. 27, the upper and lower gear driving shafts 521 and 531 and the pinion rotation shaft 551a are disposed to be perpendicular to each other.

As shown in FIG. 28, the latch coupling plate unit 555 includes a push button through-groove 555a and a pinion rotation shaft 551a through-groove, and the coupling body units 624 and 634 of the detachable fixing latch unit 600 are coupled to each other.

The detachable fixing latch unit 600 in accordance with the embodiment of the present invention is configured to attach and detach the protection cover body unit 400a to and from the module, and the protection cover holding units 511, 512, 513, and 514 are formed on the protection cover coupling plate 510 in order to reinforce the coupling restraint of the detachable fixing latch unit 600. To this end, the detachable fixing latch unit 600 includes a push button unit 610, a latch medium unit 620, and a latch unit 630, as shown in FIGS. 29 to 34.

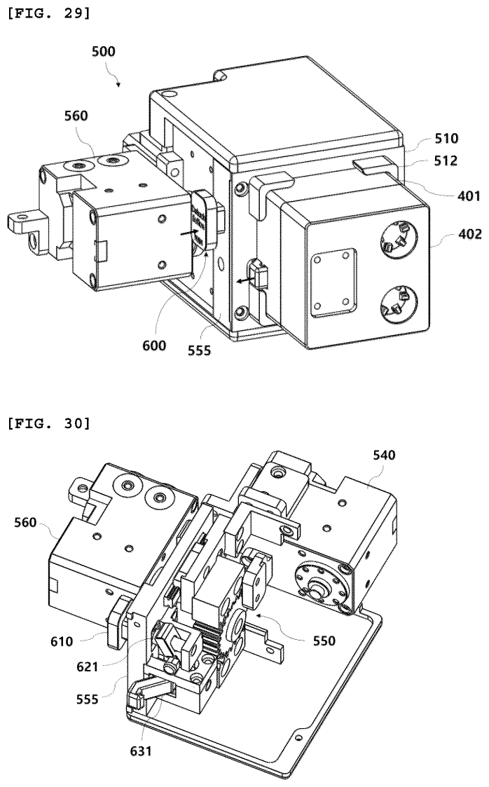

As shown in FIGS. 29 and 30, the push button unit 610 is inserted into and coupled to the push button through-groove 555a of the latch coupling plate unit 555 so that an external force parallel to the pinion rotation shaft 551a is applied. In this case, when an external force is applied, the latch coupling restraint of the protective cover body unit 400a is released, and when the external force is removed, the latch coupling restraint is performed.

As an external force is applied to the push button 610, the latch intermediate unit 620 rotates at a predetermined angle with respect to the rotation shaft 625. To this end, the latch intermediate unit 620 includes a latch intermediate body unit 621, a movement central shaft 622a, a rotation central shaft 622b, first and second springs 623a and 623b, a coupling body unit 624, and a rotation shaft 625.

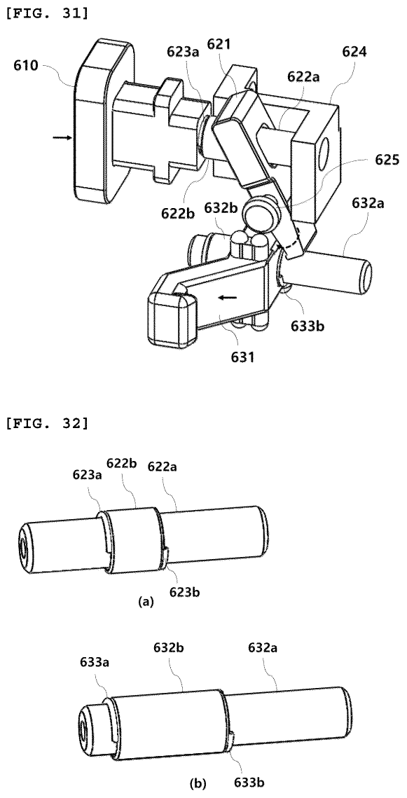

As shown in FIG. 31, the latch intermediate body unit 621 has an insertion groove into which the first central shaft 622a is inserted. The latch intermediate body unit 621 is elastically supported by a second central shaft 622b. As illustrated in FIG. 31, the second central shaft 622b may be inserted into the first central shaft 622a, and first and second springs 623a and 623b may be disposed at both ends of the second central shaft 622b to elastically support the latch intermediate body unit 621. Accordingly, when an external force is applied to the push button unit 610, the second central shaft 622b pushes the latch intermediate body unit 621 along the first central shaft 622a, and accordingly, the latch intermediate body unit 621 is rotated by a predetermined angle with respect to the rotation shaft 625. Meanwhile, when the external force applied to the push button unit 610 is removed, the external force returns to its original position by elasticity.

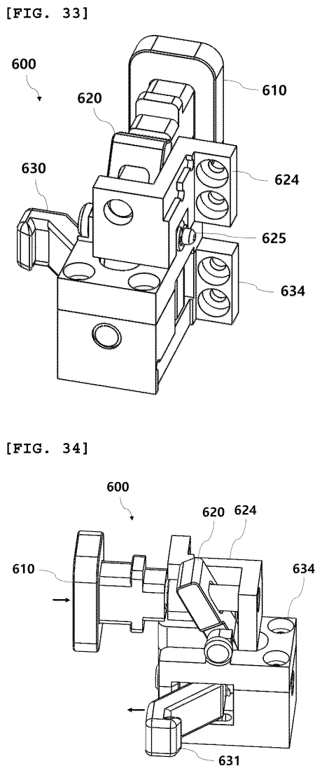

The coupling body unit 624 shown in FIG. 33 is coupled to the latch coupling plate unit 555 and has an insertion hole into which the first central shaft 622a is inserted.

As shown in FIG. 31, the latch unit 630 is interlocked with the rotation of the latch coupling plate unit 555 and elastically moves horizontally along the first central shaft 632a, thereby releasing the restraint of the latch body unit 631. To this end, the latch unit 630 includes a latch body unit 631, first and second central axes 632a and 632b, first and second springs 633a and 633b, and a coupling body unit 634.

The latch body unit 631 is inserted into the first central shaft 632a and elastically moves horizontally along the first central shaft 632a. As illustrated in FIG. 32, the second central shaft 632b is inserted into the first central shaft 632a, and first and second springs 633a and 633b are disposed at both ends of the second central shaft 632b to elastically support the latch body unit 631. Accordingly, when the latch intermediate body unit 621 is elastically rotated at a predetermined angle, the latch intermediate body unit 621 applies force to the latch body unit 631 according to the elastic rotation, and the latch body unit 631 is elastically moved horizontally along the first central shaft 632a. When the external force is removed, all are returned to their original positions by elasticity.

As shown in FIG. 33, the coupling body 634 is coupled and fixed to the latch coupling plate unit 555, and has an insertion hole into which the first central shaft 632a is inserted.

As shown in FIG. 34, when an external force is applied to the push button 610, the latch body unit 631 is elastically and horizontally moved in a direction opposite to a direction in which the external force is applied, thereby releasing the restraint of the latch body unit 631 from the protection cover body unit 400a.

In describing the present invention, descriptions of matters apparent to the prior art and the skilled person in the art may be omitted, and descriptions of elements (methods) and functions omitted may be fully referred to without departing from the technical spirit of the present invention. In addition, the above-described components of the present invention have been described for convenience of description of the present invention, but the components not described herein may be added within a range that does not deviate from the technical spirit of the present invention.

The description of the feature and the function of each of the above-described components is separately described for convenience of description, but any one feature and function may be integrated into other components or may be implemented in a more subdivided manner as necessary.

As described above, the present invention has been described with reference to an embodiment of the present invention, but the present invention is not limited thereto, and various modifications and applications are possible. That is, it will be easily understood by a skilled person in the art that many modifications are possible without departing from the gist of the present invention. In addition, it should be noted that when it is determined that a detailed description of a known function related to the present invention and a feature thereof or a coupling relation to each feature of the present invention may unnecessarily obscure the gist of the present invention, the detailed description thereof will be omitted.

1. An apparatus for preventing buckling of a flexible surgical instrument, the apparatus comprising:
a master driving unit for receiving a driving command from a user;
and a feeder unit for feeding surgical instruments according to whether to interlock with the master driving unit or not interlock with the master driving unit, and feeding the surgical instruments supplied from the master driving unit so as to prevent the surgical instruments from buckling by means of the feeding control command.

2. The apparatus for preventing buckling of a flexible surgical instrument of claim 1,
further comprising a connection support mechanically connecting and supporting the master driving unit and the feeder unit, wherein the feeder unit includes a plurality of rollers disposed at an end of the connection support unit spaced apart from the master driving unit by a predetermined distance and parallel to each other on the plurality of roller driving shafts, and the plurality of rollers are disposed near an affected area to prevent buckling of the surgical instrument by translating and rotating the surgical instrument.

3. The apparatus for preventing buckling of a flexible surgical instrument of claim 1,
wherein the feeder unit comprises: a plurality of roller driving shafts arranged in parallel to each other; a plurality of roller rotation driving units to translate the surgical instrument by rotating the plurality of driving shafts in different directions while being interlocked with each other by a gear; and a roller horizontal movement driving unit to generate driving force to horizontally move the plurality of roller driving shafts in different directions and transfer the driving force to the driving shafts to rotate the surgical instrument.

4. The apparatus for preventing buckling of a flexible surgical instrument of claim 3,
further comprising: a coupling module unit for coupling the roller driving shaft and the driving shaft to each other to able to be attached to and detached from each other in a horizontal direction to prevent mechanical restraint.

5. The apparatus for preventing buckling of a flexible surgical instrument of claim 3,
wherein the plurality of roller rotation driving units rotate the plurality of roller driving shafts in different directions by interlocking operation of one motor and one gear.

6. The apparatus for preventing buckling of a flexible surgical instrument of claim 3,
wherein the driving shaft of the roller rotation driving unit is disposed on the roller driving shaft to transmit a rotational driving force so that the roller driving shaft rotates in different directions, and the driving shaft of the roller horizontal movement driving unit is disposed perpendicular to the driving shaft of the roller rotation driving unit to transmit a horizontal movement driving force to the driving shaft so that the roller driving shaft horizontally moves in different directions.

7. An apparatus for preventing buckling of a flexible surgical instrument, the apparatus comprising:
a feeder driving unit for generating driving force to translate and rotate the surgical instrument;
a driving shaft coupling module unit which is first-coupled to the driving shaft of the feeder driving unit in a horizontal direction to be mechanically detachable;
a roller module unit which is second-coupled to the coupling shaft of the driving shaft coupling module unit in a horizontal direction to be mechanically detachable;
and an elasticity providing unit for providing elasticity to maintain mechanical coupling coupling between the first and second couplings which are mutually restrained to be mechanically detachable.

8. The apparatus for preventing buckling of a flexible surgical instrument of claim 7,
wherein a translation gear shaft of the feeder driving unit for translating the surgical instrument and a rotation gear shaft of the feeder driving unit for rotating the surgical instrument are disposed perpendicular to each other, and the surgical instrument is disposed parallel to the rotation gear shaft and disposed in a space between the plurality of rollers of the roller module unit.

9. The apparatus for preventing buckling of a flexible surgical instrument of claim 7,
wherein the feeder driving unit comprises: a plurality of roller rotation driving units generating rotation driving force to rotate a plurality of gear driving shafts disposed in parallel to each other in different directions through gear engagement to rotate the plurality of roller rotation shafts mechanically coupled to the plurality of gear driving shafts in different directions, thereby translating the surgical instrument; and a roller horizontal movement driving unit generating horizontal movement driving force to horizontally move the plurality of gear driving shafts in different directions by being disposed perpendicular to the plurality of gear driving shafts to horizontally move the plurality of gear driving shafts in different directions, thereby rotating the surgical instrument by horizontally moving the plurality of roller rotation shafts mechanically coupled to the plurality of gear driving shafts in different directions.

10. The apparatus for preventing buckling of a flexible surgical instrument of claim 9,
wherein the plurality of roller rotation driving units comprise a first roller rotation driving unit first coupled to the driving shaft coupling module unit in a horizontal direction and a second roller rotation driving unit disposed in parallel with the first roller rotation driving unit and second coupled to the driving shaft coupling module unit in a horizontal direction.

11. The apparatus for preventing buckling of a flexible surgical instrument of claim 10, wherein the first roller rotation driving unit comprises: a first gear driving shaft configured to rotate according to the rotation driving force; a first gear unit configured to rotate according to rotation of the first gear driving shaft; a bearing unit configured to rotate the first gear unit while being relatively restrained from rotation, and to horizontally move together with the first gear driving shaft in a first direction according to transmission of the first direction horizontal movement driving force; and a roller rotation motor unit configured to generate the rotation driving force to rotate the first gear driving shaft. The apparatus for preventing buckling of a flexible surgical instrument of claim 10, wherein the first roller rotation driving unit comprises: a first gear driving shaft configured to rotate according to the rotation driving force; a first gear unit configured to rotate according to rotation of the first gear driving shaft; a bearing unit configured to rotate the first gear unit while being relatively restrained from rotation, and to horizontally move together with the first gear driving shaft in a first direction according to transmission of the first direction horizontal movement driving force; and a roller rotation motor unit configured to generate the rotation driving force to rotate the first gear driving shaft.

12. The apparatus for preventing buckling of a flexible surgical instrument of claim 11,
wherein the second roller rotation driving unit includes: a second gear unit gear-meshed with the first gear unit to rotate in a direction different from a rotation direction of the first gear unit; a second gear driving shaft rotating in the same direction as the rotation direction of the second gear unit; a bearing unit relatively rotatably restrained while rotating the second gear unit to horizontally move in a second direction together with the second gear driving shaft according to transmission of the second direction horizontal movement driving force; and a motor coupler unit having a driving shaft insertion movement groove into which the second gear driving shaft is inserted to horizontally move in the second direction.

13. The apparatus for preventing buckling of a flexible surgical instrument of claim 12,
wherein the roller horizontal movement driving unit comprises: a pinion unit configured to rotate with respect to a pinion rotation shaft by the horizontal movement driving force; a first rack unit engaged with a gear of the pinion unit to generate the first direction horizontal movement driving force; a second rack unit engaged with a gear of the pinion unit to generate the second direction horizontal movement driving force; and a roller horizontal movement motor unit configured to generate the horizontal movement driving force to rotate the pinion rotation shaft.

14. The apparatus for preventing buckling of a flexible surgical instrument of claim 13,
further comprising: a first driving shaft horizontal movement coupling unit coupled to the first rack unit and the bearing unit of the first roller rotation driving unit to transmit the first direction horizontal movement driving force to the bearing unit of the first roller rotation driving unit and to restraint rotation of the bearing unit of the first roller rotation driving unit; a second driving shaft horizontal movement coupling unit coupled to the second rack unit and the bearing unit of the second roller rotation driving unit to transmit the second direction horizontal movement driving force to the bearing unit of the second roller rotation driving unit and to restrain rotation of the bearing unit of the second roller rotation driving unit; a first rail unit to allow the first driving shaft horizontal movement coupling unit to move in a horizontal direction; and a second rail unit to allow the second driving shaft horizontal movement coupling unit to move in a horizontal direction.

15. The apparatus for preventing buckling of a flexible surgical instrument of claim 7,
wherein the driving shaft coupling module unit includes: a first coupling disk unit that is first coupled with the first gear driving shaft of the feeder driving unit in a horizontal direction and is second coupled with the first roller rotation shaft of the roller module unit in a horizontal direction; and a second coupling disk unit that is disposed in parallel with the first driving shaft, is first coupled with the second gear driving shaft of the feeder driving unit in a horizontal direction, and is second coupled with the second roller rotation shaft of the roller module unit in a horizontal direction.

16. The apparatus for preventing buckling of a flexible surgical instrument of claim 15,
wherein the roller module unit comprises: a first surgical instrument roller unit which is second-coupled to one side of the first coupling disk unit and is elastically supported from the first elastic unit of the elasticity providing unit and the other side thereof; and a first surgical instrument roller unit which is second-coupled to one side of the second coupling disk unit and is elastically supported from the second elastic unit of the elasticity providing unit and the other side thereof.

17. The apparatus for preventing buckling of a flexible surgical instrument of claim 16,
wherein the plurality of rotation shafts of the roller module unit and the plurality of gear driving shafts of the feeder driving unit are mechanically restrained to be detachable on a shaft by the first and second coupling disk units, respectively, to be first and second coupled to each other, such that translation or rotation of the surgical instrument is performed.

18. The apparatus for preventing buckling of a flexible surgical instrument of claim 7,
further comprising a surgical instrument guide unit for guiding the surgical instruments supplied from the master driving unit to spaces between the plurality of rollers of the roller module unit.

19. The apparatus for preventing buckling of a flexible surgical instrument of claim 18,
wherein the surgical instrument guide unit comprises a guide body having a surgical instrument penetration hole, a body coupling unit coupling and fixing the guide body to the driving shaft coupling module unit and the elasticity providing unit, respectively, and a tubular guide unit guiding the surgical instrument.

20. The apparatus for preventing buckling of a flexible surgical instrument of claim 7,
further comprising a detachable fixing latch unit configured to release the restraining of the driving shaft coupling module unit, to which the latch is mechanically restrained and fixed, by being moved by an external force, and configured to mechanically restrain and fix the driving shaft coupling module unit by returning to its original position as the external force is removed.

21. The apparatus for preventing buckling of a flexible surgical instrument of claim 20,
wherein the driving shaft coupling module further includes a protection cover unit protecting the first and second coupling disk units and having a latch insertion groove into which the latch is inserted and restrained and fixed, and the feeder driving unit includes a protection cover coupling plate in which an L-shaped protection cover holding unit is formed to hold a plurality of corners of the protection cover unit in order to assist the attachment/detachment restraining and fixing of the attachment/detachment fixing latch unit and having a latch penetration groove, and a latch coupling plate unit coupled and fixed to the attachment/detachment fixing latch unit as a housing of the feeder driving unit together with the protection cover coupling plate.

22. The apparatus for preventing buckling of a flexible surgical instrument of claim 20,
wherein the detachable fixing latch unit comprises: a push button unit to which an external force is applied; a latch medium unit which rotates at a predetermined angle about a rotation shaft according to elastic pressing of the push button unit; and a latch unit which elastically moves in a horizontal direction in conjunction with rotation of the latch medium unit.

23. The apparatus for preventing buckling of a flexible surgical instrument of claim 22,
wherein the latch unit is elastically moved horizontally in a direction opposite to the direction of the external force to release the driving shaft coupling module unit from the restraint.

24. The apparatus for preventing buckling of a flexible surgical instrument of claim 22,
wherein the latch medium unit is disposed to be inclined at a predetermined angle from an initial state in which the external force is removed.

25. The apparatus for preventing buckling of a flexible surgical instrument of claim 15,
wherein a restraint disk is formed on one side of the first and second coupling disk units in a circumferential direction, and a restraint disk insertion groove is formed on the other side in the circumferential direction.
